(19) United States
(12) Patent Application Publication
(10) Pub. No.: US 2021/0018348 A1
(43) Pub. Date: Jan. 21, 2021

(54) SYSTEMS AND METHODS FOR MEASURING INJECTED FLUIDS

(71) Applicant: Osprey Medical, Inc., Minnetonka, MN (US)

(72) Inventors: Dale Brady, New Brighton, MN (US); Rodney L. Houfburg, Prior Lake, MN (US); Steve Rathjen, South Lake Tahoe, CA (US); Matthew M. Burns, Orono, MN (US); Alexander Frederick Dietz, Minneapolis, MN (US)

(73) Assignee: Osprey Medical, Inc., Minnetonka, MN (US)

(21) Appl. No.: 16/931,664

(22) Filed: Jul. 17, 2020

Related U.S. Application Data
(60) Provisional application No. 62/875,859, filed on Jul. 18, 2019.

(51) Int. Cl. G01F 1/56; G01F 9/00

(52) U.S. Cl. CPC G01F 1/56; G01F 9/003

(57) ABSTRACT

A fluid collection and measurement container includes a first wall and a second wall disposed opposite the first wall. The second wall is secured to the first wall and the first wall and the second wall define an expandable volume therebetween. A first electrolytic element is disposed on the first wall and a second electrolytic element is disposed on the second wall. A first terminal is connected to the first electrolytic element and a second terminal is connected to the second electrolytic element.

### CROSS-REFERENCE TO RELATED APPLICATIONS

This application claims priority to and the benefit of U.S. Provisional Patent Application Ser. No. 62/875,859, filed Jul. 18, 2019, entitled “Systems and Methods for Measuring Fluid Flow,” the disclosure of which is hereby incorporated by reference herein in its entirety

### INTRODUCTION

There are numerous instances in the diagnostic, prophylactic, and treatment practice of medicine wherein an agent, medicant, or medium is preferably delivered to a specific site within the body, as opposed to a more general, systemic introduction. One such exemplary occasion is the delivery of contrast media to coronary vasculature in the diagnosis (i.e., angiography) and treatment (i.e., balloon angioplasty and stenting) of coronary vascular disease.

### SUMMARY

This summary is provided to introduce a selection of concepts in a simplified form that are further described below in the Detailed Description. This summary is not intended to fully identify key features or essential features of the claimed subject matter, nor is it intended to describe each and every disclosed example or every implementation of the claimed subject matter, as well as is not intended to be wholly used as an aid in determining the scope of the claimed subject matter. Many other novel advantages, features, and relationships will become apparent as this description proceeds. The figures and the description that follow more particularly exemplify illustrative examples.

In one aspect, the technology relates to a fluid collection and measurement container having: a first wall; a second wall disposed opposite the first wall, wherein the second wall is secured to the first wall and wherein first wall and the second wall define an expandable volume therebetween; a first electrolytic element disposed on the first wall; a second electrolytic element disposed on the second wall; a first terminal connected to the first electrolytic element; and a second terminal connected to the second electrolytic element.

In another aspect, the technology relates to a system for measuring an amount of a fluid medium, the system including: a power injector for providing automated ejection of the fluid medium; a delivery catheter for providing delivery of at least a first portion of the fluid medium into the patient, during use; at least two flow controllers selectively fluidically coupled to the power injector; a fluid flow control apparatus fluidly coupled between the power injector, the delivery catheter, and the at least two flow controllers, wherein the fluid flow control apparatus, during use, provides fluid diversion of at least a second portion of the fluid medium, the second portion of the fluid medium being diverted away from the delivery catheter, and wherein an amount of diversion of the second portion of fluid is dependent on a selection of one of the at least two flow controllers, wherein the at least two flow controllers are characterized by applying differing resistances to the second portion of the fluid medium; a collection container for receiving the second portion of the fluid medium; first sensor capable of measuring an elected amount of the fluid medium ejected by the power injector; and a second sensor disposed on the collection container, wherein the second sensor comprises a first part disposed on a first wall of the collection container, a second part disposed on a second wall of the collection container, and wherein the second sensor detects a change in capacitance between the first part and the second part.

In another aspect, the technology relates to a method of determining a volume of a fluid in a container having a first wall and a second wall adjacent the first wall, the method including: sending, to a processor, a first signal from a capacitor disposed on the container, wherein the first signal is associated with a first separation distance between the first wall of the container and the second wall of the container; receiving the fluid in the container, wherein receiving the fluid in the container changes a separation distance between the first wall of the container and the second wall of the container; and sending, to the processor, a second signal from the capacitor, wherein the second signal is different than the first signal, and wherein the second signal is associated with a second separation distance between the first wall of the container and the second wall of the container.

In another aspect, the technology relates to a method of calculating a volume of a fluid received in a container having a first wall and a second wall adjacent the first wall, the method including: receiving a first signal from a capacitor disposed on the container, wherein the first signal is associated with a first separation distance between the first wall of the container and the second wall of the container; receiving a second signal from the capacitor, wherein the second signal is different than the first signal, and wherein the second signal is associated with a second separation distance between the first wall of the container and the second wall of the container; and processing the first signal and the second signal to calculate the volume of the fluid received in the container.

### BRIEF DESCRIPTION OF THE DRAWINGS

There are shown in the drawings, examples of which are presently preferred, it being understood, however, that the technology is not limited to the precise arrangements and instrumentalities shown.

FIGS. 1A and 1B depict a examples of fluid diversion systems.

FIG. 2 depicts an example of a fluid diversion system in combination with a measurement system.

FIG. 3 depicts an example of a diversion unit that may be utilized in a fluid measurement system.

FIG. 4 depicts an example of a flow sensor that may be utilized in a fluid measurement system.

FIG. 5 depicts an example of a fluid collection and measurement system.

FIG. 5A depicts an alternative example of a collection and measurement container that may be utilized in a fluid measurement system.

FIGS. 6A and 6B depict front and side views of another example of a collection and measurement container that may be utilized in a fluid measurement system.

FIGS. 7A and 7B depict details regarding operation of a known capacitor.

FIGS. 8A-8C depict the collection and measurement container of FIGS. 6A and 6B at various filled conditions.

FIGS. 9A-9D depict alternative examples of sensing devices utilized on collection containers.

FIGS. 10A and 10B depict front and exploded perspective views of an alternative example of a sensing device utilized on a collection container.

FIGS. 11A-11C depict alternative examples of collection containers.

FIGS. 12A-12B depict alternative examples of collection containers.

FIG. 13 depicts a method of determining a volume of a fluid in a container.

FIG. 14 depicts a method of calculating a volume of a fluid in a container.

FIG. 15 depicts one example of a suitable operating environment in which one or more of the present examples may be implemented.

### DETAILED DESCRIPTION

This disclosure pertains to systems, devices, and methods used to modulate or alter the delivery of a substance, such as radiopaque contrast, to a delivery site and/or systems, devices, and methods that may be used to measure or otherwise make quantitative assessments of a medium delivered to a delivery site. More specifically, it is an intention of the following systems, devices, and methods to alter the injection of media to a vessel, vascular bed, organ, and/or other corporeal structures so as optimize the delivery of media to the intended site. It is also an intention of the present technology to provide a way to assess the amount of medium injected, in light of the modulation of the injection. Such systems, devices, and methods described provide measurement of injections to closely monitor the amount of medium injected, while reducing inadvertent or excessive systemic introduction of the media through alteration of the medium flow path. Another aim of this disclosure is to describe systems, devices and methods that may accommodate the use of a power injector while modulating and measuring medium delivered to a patient. Other benefits of the described systems, devices, and methods will be apparent to a person of skill in the art.

The description, as well as the devices and methods described herein, may be used in modulating and/or monitoring medium delivery to the coronary vasculature in prevention of toxic systemic effects of such an agent. One skilled in the art, however, would recognize that there are many other applications wherein the controlled delivery and/or quantitative assessment of a media to a specific vessel, structure, organ or site of the body may also benefit from the devices and methods disclosed herein. For simplicity, these devices and methods may be described as they relate to contrast media delivery modulation and/or measurement. As such, they may be used in the prevention of Contrast Induced Nephropathy (CIN); however, it is not intended, nor should it be construed, so as to limit the use to this sole purpose. Exemplary other uses may include the delivery, injection, modulation, or measurement of: cancer treatment agent to a tumor, thrombolytic to an occluded artery, occluding or sclerosing agent to a vascular malformation or diseased tissue; genetic agent to a muscular bed, neural cavity or organ, emulsion to the eye, bulking agent to musculature and/or sphincter, imaging agent to the lymphatic system, antibiotics to an infected tissue, supplements in the dialysis of the kidney, to name but a few.

The terms medium (media), liquid, agent, substance, material, medicament, and the like, are used generically herein to describe a variety of fluid materials that may include, at least in part, a substance used in the performance of a diagnostic, therapeutic or/and prophylactic medical procedure and such use is not intended to be limiting.

CIN is a form of kidney damage caused by the toxic effects of dyes (e.g., radiopaque contrast media) used, for example, by cardiologists to image the heart and its blood vessels during commonly performed heart procedures, such as angiography, angioplasty, and stenting. In general, the dye is toxic and is known to damage kidneys. Although most healthy patients tolerate some amount of the “toxicity,” patients with poorly or non-functioning kidneys may suffer from rapidly declining health, poor quality of life, and significantly shortened life expectancy. Potential consequences of CIN include:

irreversible damage to the kidneys, longer hospital stays, increased risk of heart disease, increased risk of long-term dialysis, and ultimately, a higher mortality risk. For patients who acquire CIN, their risk of dying remains higher than others without CIN, and this risk can continue up to five years after their procedure. CIN has a significant economic burden on the healthcare system and currently there is no treatment available to reverse damage to the kidneys or improper kidney performance, once a patient develops CIN.

To date, there have been attempts in reducing the toxic effects of contrast media on patients who undergo procedures involving dyes, especially those patients who are at high risk for developing CIN. Some of these efforts have been to: change the inherent toxicity (of a chemical or molecular nature) of the dyes, reduce the total amount of contrast agent injected (through injection management and/or dye concentration), and remove media through coronary vasculature isolation and blood/contrast agent collection systems, to name a few. These methods and devices used in the control of the toxic effects of contrast agents have had their inherent compromises in effectively delivering a contrast media specifically to a target site while minimizing the systemic effects. As an example, changing the composition of a dye and/or injection concentration may help reduce a contrast agent's inherent toxicity at the expense of the contrast agent's ability to perform its intended function (e.g., visualization of vasculature). Conversely, the ability to “collect” contrast agent laden blood “downstream” from the visualization site may ensure visualization, but requires the complexity of placement and operation of a collection system.

Other attempts to manage the amount of contrast agent delivered to a patient have employed automated, powered (versus manual, syringe-injected) contrast media injection systems. Close monitoring and control of the total quantity of contrast agent injected may have a positive impact in reducing the incidence of CIN. However, these injection systems are expensive (including capital equipment and disposables), cumbersome to use within a cath lab, and take additional time and expertise to set up and operate properly. Improper use could negate any benefits seen by better management of the quantity of the contrast agent delivered to a patient, and the additional time required to set up such a system may also add significant complexity to a procedure. The devices and methods described herein may measure or otherwise quantitatively assess the amount of medium injected or delivered to a delivery site using a relatively fast, simple, economical, and safe system.

In addition, end users may have varied different needs, and as such, the various components and methods described herein for measurement, modulation, and diversion (i.e., for example, a reservoir for reuse of the medium) may be used in part, or whole, to address these needs. As an example, one user may only want to measure an injection (while not measuring a saline flush); another user may want to employ a modulator and measurement, while not capturing the diverted medium for reuse (medium wasted); further, another user may want to employ measurement and a reservoir for reuse, but would prefer to use their existing system for reuse capture. These are merely a small list of the various needs addressed by combining different components of the described examples herein, and they should be viewed as exemplary and not limiting. Further, the use of an injector has been described and as such it could be a syringe and/or a power injector (e.g., Acist CVi Injector). Construction of examples described herein may vary depending on the injector; however, the principals of the examples may remain the same.

The examples described herein may include various elements or components to measure and/or detect a displacement of a plunger within a chamber, such as a syringe, or an automated injector. And, with the detection of a positional relationship of a plunger within a chamber, a user may explicitly or implicitly determine a volume of media that may have been ejected from a chamber. Some of the examples described may include various components to detect or sense positional relationship of the plunger/piston and the chamber. Linear encoders, inductive sensors, capacitive touch sensors (with metal actuator in plunger), ultrasonic emitters/receivers, pressure sensors, optical encoders (with fine pitch slots and light source), strain gauges (i.e., to measure weight), electromagnetic emitters/receivers (e.g., navigational systems) are alternative technologies contemplated for the use of measuring an injection delivered from an injector to a patient, with or without measuring a “diversion” reservoir. Other alternative examples capable of identifying positional relationships of a plunger and chamber (and changes thereof) may include, without limitation, the following technologies. A Hall sensor (coiled wire along syringe axis) may be placed on, or in proximity to, the chamber with a magnet attached to the plunger (so as to act as a variable proximity sensor). Multiple low sensitivity Hall sensors may be disposed along the chamber of the syringe with a magnet attached to the plunger. Still other examples of systems utilizing multiple Hall sensors are described herein. Laser light may be emitted and detected to determine a positional relationship of the plunger along the chamber axis. An absolute encoder may be used to “read” the direct displacement of the plunger. Many of these systems described herein include at least a two part, or potion, of a sensing system One part may be used to send or cause the creation of a signal (or change), and the second part may be used to read, sense, or measure a difference in a signal (or change). Typically, in the many of the examples described herein, one of the components (i.e., part, portion, etc.) of measurement may be associated, attached to, or in the proximity with the plunger of an injector; whereas, the at least second part (i.e., component, portion, etc.) may be attached to, associated with, or in the proximity of the injector housing. This application references “Contrast Diversion and Measurement,” filed Jun. 30, 2018, as U.S. Ser. No. 16/024,768, published as U.S. Patent Publication No. 2018/0318495, the disclosure of which is hereby incorporated by reference herein in its entirety.

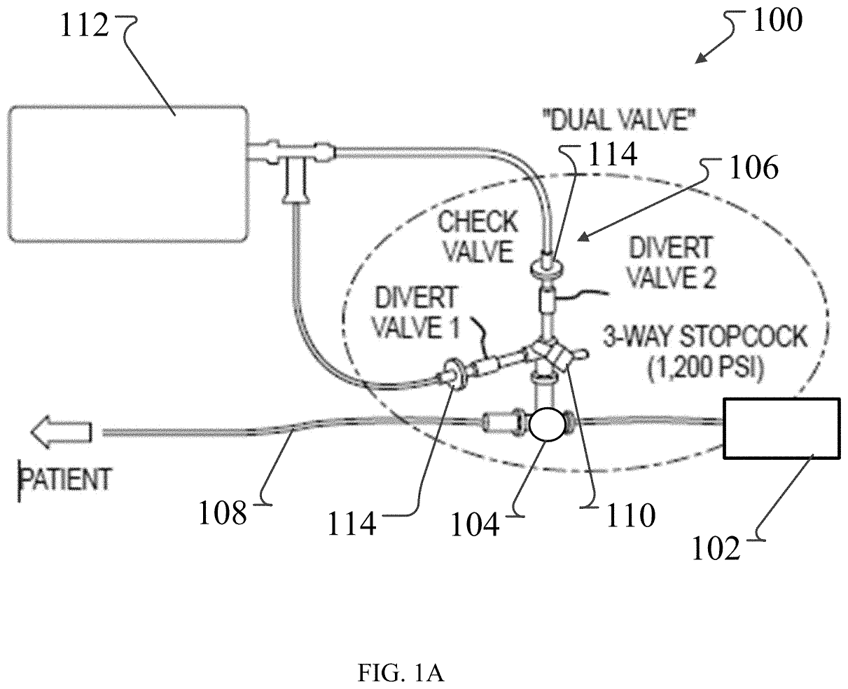

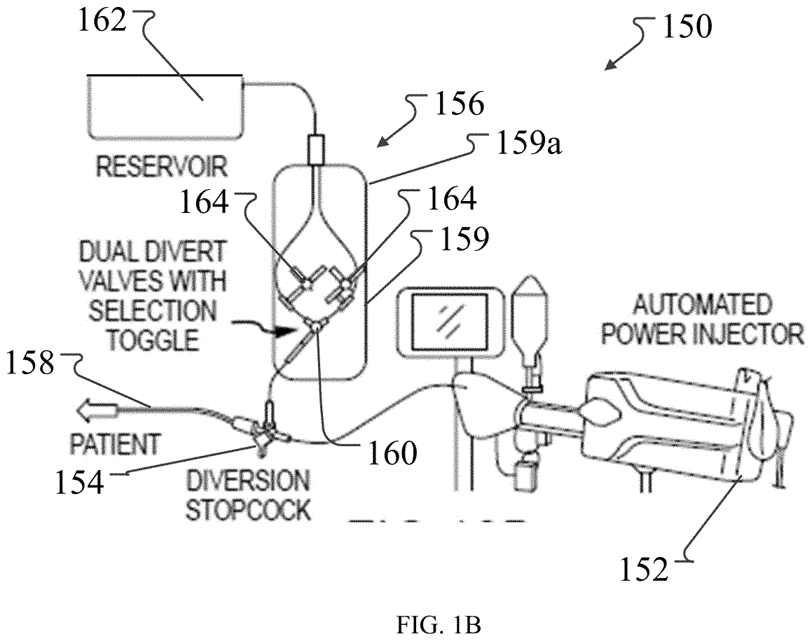

FIGS. 1A and 1B depict examples of fluid diversion systems. Although FIG. 1A is generally described first, components and functionalities common to systems depicted in both FIGS. are also described. Further, components depicted within one of the two systems may also be incorporated into the other of the two systems. FIG. 1A depicts a first example of a fluid measurement system 100. The system 100 is connected to an injector 102 that is configured to deliver medium to a patient. The system 100 is a so-called “dual valve” configuration that utilizes two diversion or divert valves (as described in more detail below). A portion of an injection is diverted from the injector 102 to, and through, diversion valve #1 (DV1) or diversion valve #2 (DV2). Each diversion valve DV1, DV2 may be a two-way (e.g., on-off) valve. In other examples, the diversion valves may be adjustable valves, the flow through which may be adjusted by adjusting a position of the valve actuator. In other examples, the valves may be variable in their response to varying increases/decreases of pressure and/or flow within their diversion pathways. In other examples the diversion valves may be fixed-position flow control devices (e.g. flow restrictors) that allow only a fixed flow rate therethrough. For clarity, however, the following description will use the term “diversion valve” or “divert valve”. A toggle 104 or stopcock may be used to open a diversion flow path 106 (containing DV1 and DV2) to divert medium ejected from the injector 102. Although two diversion valves DV1 and DV2 are depicted, it is contemplated that multiple divert valves (e.g., three, four, or more) may be utilized to address a great number of user preferences. Each diversion valve utilized may correspond to a different diversion profile. Further, diversion valves DV1 and DV2 are depicted as being configured in a parallel fashion. However, one skilled in the art would recognize that some valves may be aligned in a serial fashion to obtain an identical or similar net result.

Regardless of the number of diversion valves utilized, each such diversion valve may accommodate a variety of injections from an injector (such as injector 102) through a variety of delivery conduits 108 while regulating the diversion of a portion of the injected fluid via the diversion flow path 106, so as to maintain a relatively constant flow injection to the patient. In such cases, the diversion valve (DV1 or DV2 in the example of FIG. 1A) may indirectly regulate the various flows/pressures of the fluid injected. The diversion valve (DV1 or DV2) may variably regulate the flow to the patient by relatively increasing resistance to diversion with increasing pressure at a selected diversion valve. However, such a configuration may not sufficiently address all situations wherein there are large differences in the pressure/flow from the injector 102, or there are large differences in the operational use to deliver a fluid at largely different rates, or through injection systems wherein the delivery conduit 108 to the patient are vastly different in structure and/or configurations, to name a few examples. A particular diversion valve (DV1 or DV2) may be selected by positioning a stopcock 110. In examples, the stopcock 100 may also be utilized to completely close off flow to the diversion flow path 106, thus obviating the need for toggle 104. FIG. 1A depicts a system 100 having two separate diversion valves DV1, DV2 and discrete check valves 114 (to maintain directional fluid flow within diversion flow path 106). In this example, the excess diverted fluid may be diverted to a reservoir 112 for storage, reuse, or disposal.

FIG. 1B depicts another example of a fluid injection and diversion system 150. In this example, the system 150 includes an automated power injector 152. A diversion stopcock 154 may allow fluid diversion to a diversion flow path 156, and away from the delivery conduit 158. A fluid flow control apparatus 159 includes a housing 159a that houses a portion of the diversion flow path 156. The flow control apparatus 159 includes a diversion stopcock 160 that directs flow to one of two alternative diversion valves 164. As noted above, flow restrictors may be utilized in lieu of the depicted diversion valves, the flow path through which is selected based on the position of the diversion stopcock 160. As with the example described above with regard to FIG. 1A, a greater number of diversion valves 164 (or flow restrictors) may be utilized and included within the housing 159a, each of which may have a different diversion profiles. Also, as with the example of FIG. 1A, diversion flow may be delivered to a reservoir 162.

A number of different uses of the fluid injection and diversion systems such as shown in FIGS. 1A and 1B are contemplated. In one example, the delivery conduits 108, 158 may include using multiple delivery conduits with each having different dimensional characteristics. As an example, a fluid may be delivered through a 4 F delivery catheter to a patient injection site utilizing a first divert valve flow pathway to perform diagnosis on a patient's vasculature. Subsequently, an 8 F catheter may be utilized with treatment devices (i.e., angioplasty, stent, atherectomy, etc.) wherein the medium may be injected to the patient utilizing a second diversion pathway, creating a different fluid flow profile injected to the patient. Utilizing different sized delivery catheters to different injection sites may also be advantageous, for example, for injections to the heart versus injections into peripheral vascular sites, or injection to one large coronary branch (e.g., the right coronary artery (RCA)) versus a different large coronary branch (e.g., left main, left coronary artery, and/or left circumflex artery). In another example, an automated injector such as shown in FIG. 1B may be utilized to maintain a constant flow and/or pressure (automated injectors generally display less variable performance than a syringe). Such automated injectors may nevertheless require adjusting the injection flow/pressure to assess different vascular sites.

Fluid measurement of an injection/diversion system 100, 150 may be performed using a number of sensors. At various locations within the systems 100, 150 depicted in FIGS. 1A and 1B, sensors may be utilized to aid in automating measurements of the injection systems 100, 150. In examples, valves (e.g., valves 104, 110, DV1, DV2, 154, 160, etc.) may incorporate or utilize a positional sensor to determine the condition (e.g., open, closed, etc.) of the particular valve. Additionally, sensors such as flow sensors, pressure transducers, or any other sensors, may be utilized to determine the conditions of the various conduits and fluid lines. In non-limiting examples, such sensors may be placed downstream of the injector, upstream of the patient, at various locations on the diversion fluid path, at the reservoir inlet, and at other locations. In another example more relevant to the system 150 of FIG. 1B, a user (e.g., doctor, technician) may change the injection setting for injection into different vascular sites, e.g., before or during operation of the automated injector. As an example, the user may select a constant flow or pressure for injection into the RCA, and select a different flow or pressure for the left coronary artery. If the flow or pressure rates differ significantly between the two sites, the user may alternatively or additionally toggle between the available diversion valves to set a different diversion profile to address each site.

Further, it is also understood that any measurement apparatuses, such as those described in U.S. Patent Publication No. 2018/0318495, referenced above, for the diversion reservoir measuring system and the injector measuring system, may also be employed in automating the measurements of fluid delivered to the patient. In addition, if the power injector depicted in FIG. 1B includes one or more features to measure fluid ejected from the injector, it may be desirable to coordinate data collection from the injector and a reservoir measurement apparatus associated with the diversion reservoir to automate the measurement of the medium injected to the patient. Systems that incorporate such further sensors, components, and functionality are described below.

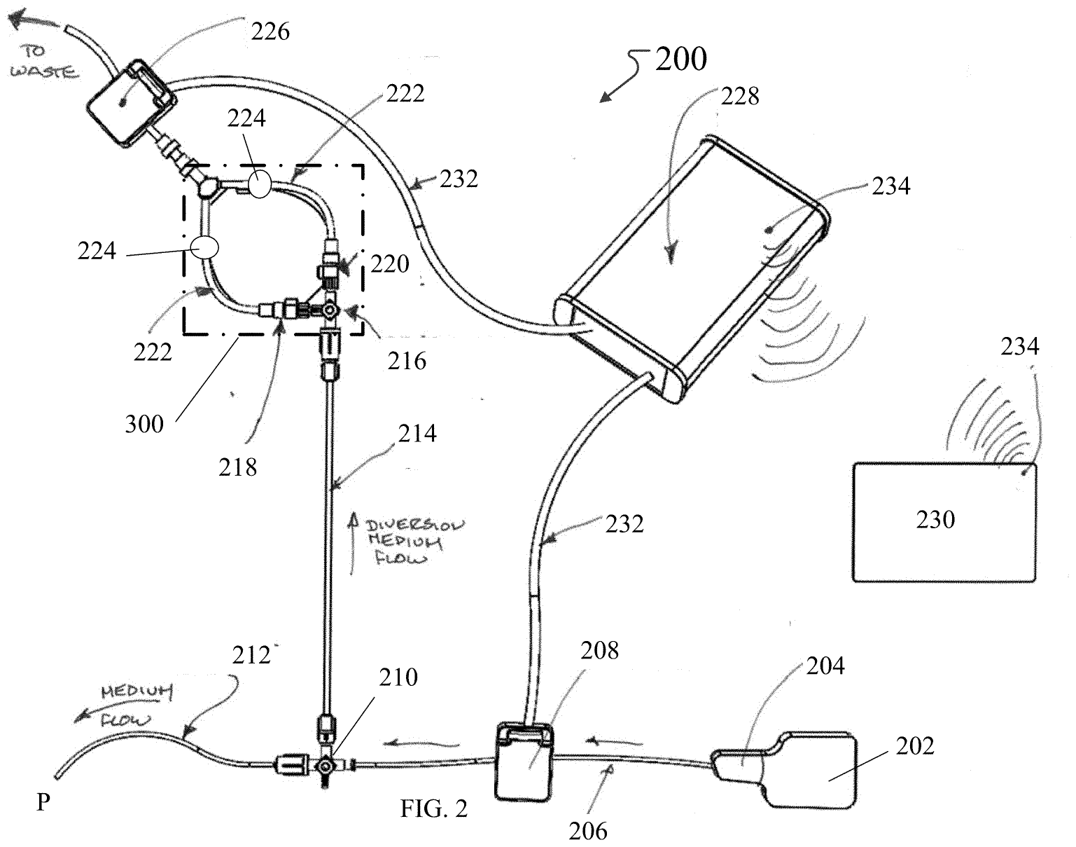

FIG. 2 depicts an example of a fluid measurement system 200 that could be used with an injection/diversion system 100, 150, as illustrated in FIGS. 1A and 1B. System 200 may modulate conditions at various locations so as to reduce the amount of medium injected into a patient P, while monitoring and measuring the total amount of medium injected to the patient P. The measuring and monitoring of the total amount of medium injected into the patient P may be helpful for a number of reasons. For example, such measuring and monitoring may inform a physician when the patient P may be at risk of systemic overload of a medium (which may cause, e.g., acute kidney injury). As shown, medium (e.g., contrast) may be supplied by a power injector 202 into the delivery system to the patient P. The injector 202 may include a medium injection chamber 204 storing sterile medium prior to ejecting the medium from the chamber 204 into the system 200 (via the discharge conduit 206). An ultrasonic injector flow sensor 208 may be disposed on the discharge conduit 206 exiting the power injector 202.

Continuing with FIG. 2 as shown, the injector flow sensor 208 may collect ultrasonic data on the flow through the discharge conduit 206 from the injector 202 and this data may be collected and/or processed to determine the volume of flow of an injection. Further, data regarding multiple injections may be acquired and stored as described herein. This may allow for generation of a summary of all injections to the patient P. This information may be tabulated or otherwise organized to be easily understood by the physician. The flow from the discharge conduit 206 may pass into a three-way valve type mechanism 210 (e.g., a stopcock). Two outlet conduits extend from the stopcock 210: a fluid pathway to the patient P via a catheter 212, and a fluid pathway to a diversion conduit 214. A flow restrictor selection toggle 216 may divert flow to one of two flow restrictors, 218, 220 or divert valves. Each flow restrictor 218, 220 may be configured to create different flow profile (and range of flows) that act to create the actual medium flow injected into the patient P. That is to say, the flow of medium to the patient P may be indirectly controlled by the diversion conduit 214, and the particular flow restrictor 218, 220 employed.

In addition, downstream of each flow restrictor 218, 220 is a diversion pathway 222 that may include a one-way (check) valve 224 to allow the medium to pass in only one direction (e.g., toward container or reservoir such as described elsewhere herein). In an alternate construction, the one-way valve may be incorporated into the divert valves 218, 220. During use, the medium from the injector 202 passes to the patient P, as well as through the diversion conduit 214 (e.g., altering the flow to the patient P). The actual amount of diversion may be controlled by selecting one of the two flow restrictors 218, 220, by positioning the toggle 216. Selection of the appropriate flow restrictor 218, 220 is dependent on the flow profile intended to be delivered to the patient P. As discussed, the selection may be based on a variety of uses including catheter systems being used, vascular sites being accessed, etc. In addition, more than two divert valves 218, 200 could be utilized depending on the injection needs.

Further describing FIG. 2, when medium is being injected to the patient P, a diversion conduit sensor 226, such as an ultrasonic flow sensor, may be used to measure the flow through the diversion conduit 214. The data from the diversion conduit sensor 226 may be collected and/or processed by a data acquisition unit 228, as described in more detail herein. Similarly, data collected by the injector flow sensor 208 may be collected and processed by the data acquisition unit 228. This data, or information derived therefrom, also may be transmitted to a processor (e.g. at the data acquisition unit 228) and then displayed at a remote display 230. Information which may be particularly valuable to be displayed to the physician may include the volume injected in a particular injection, total volume injected to the patient over the course of an entire treatment, an indication of the valve 218, 220 being utilized, etc. As discussed previously, there may be a variety of configurations for the sensors and the data collection, processing, and display (e.g., wireless vs. wired communication, component of the power injector machine vs. independent system, etc.). In the depicted configuration, the data acquisition unit 228 is connected to the injector flow sensor 208 and the diversion conduit sensor 226 via wired connections 232. Each of the data acquisition unit 228 and the display 230 include a transceiver 234 for wireless communication. In another configuration, the data acquisition unit 228 and/or display 230 could be a component of the injector 202.

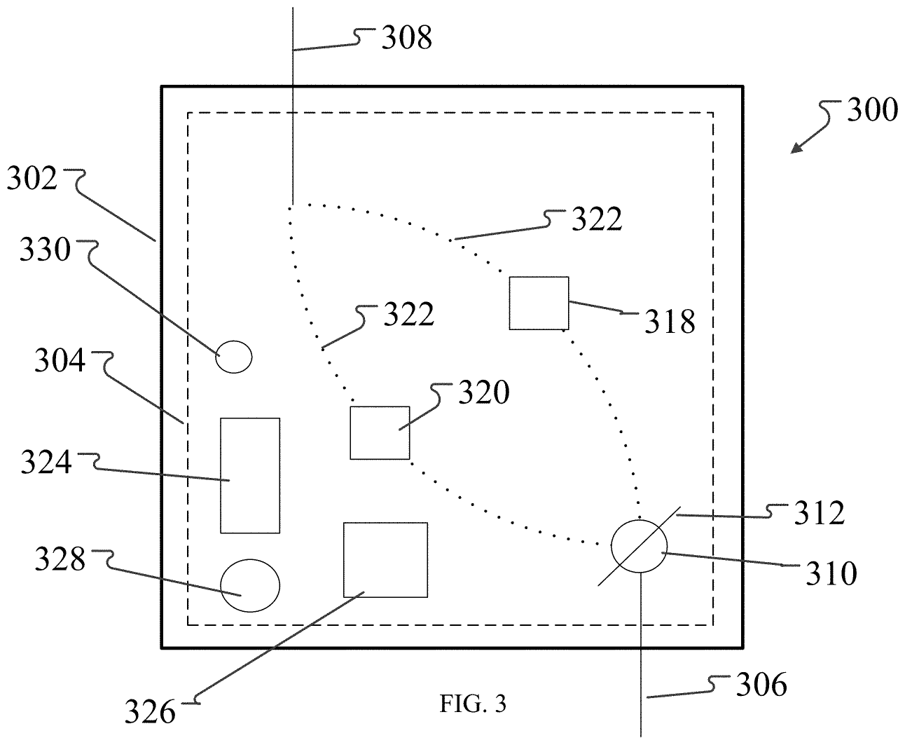

Additional or alternative features of the system 200 are contemplated. For example, the diversion pathway 214 may include a diversion or fluid flow control apparatus, which is depicted in FIG. 2 by dot-dash line 300, and described further with regard to FIG. 3. The diversion unit 300 may include a housing 302 that contains a printed circuit board 304 on which are mounted a number of components to improve unit performance and the user experience. As the components are mounted to the circuit board 304, specific connections between components are not depicted, but would be apparent to a person of ordinary skill in the art.

The diversion unit 300 includes a diversion pathway connection 306 that may be connected to the diversion pathway (e.g., the tubing or conduit forming said pathway). Additionally, the diversion unit 300 may also include a waste pathway connection 308 that may be connected to a conduit that terminates at a collection reservoir, described elsewhere herein. The diversion pathway connection 306 terminates at a selection valve 310, which may be manually activated by a lever 312 that projects from the housing 302. In another example, the selection valve 310 may be motorized and controlled via a controller disposed elsewhere on the housing 302 or in the system. The position of the selection valve 310 dictates which the diversion pathway and thus, which flow control component 318, 320 (which may include an integral check valve or be associated with a discrete check valve) is utilized. A plurality of LEDs or other light emitting elements (depicted by dotted lines 322) may be disposed proximate each of the diversion pathways and may be illuminated based on the position of the selection valve 310. The plurality of LEDs may be disposed within or on the housing 302 and provide a visual indication of the diversion pathway being utilized. In an example, the LEDs 330 may be disposed on an overlay on an exterior of the housing 302. Each diversion pathway is connected downstream to the waste pathway connection 308. Additional components within the housing 302 may include a processor 324, which may be used to process various signals sent to and from various components, sensors, valves, etc. (both within and external to the diversion unit 300). A transceiver 326 may be utilized to communicate with a data acquisition unit and/or display as described elsewhere herein. A battery 328 may provide power to the various components. The battery 328 may be alkaline, which may enable easier disposal. The battery 328 may be replaceable, such that the entire diversion unit 300 is reusable, or the entire unit 300 may be disposable. In other examples, the battery may be rechargeable. One or more buttons, switches, or control elements 330 may be disposed on the housing 302 to enable control of the unit 300. Such control may include selecting a position of the selector valve 310, pausing or stopping calculation of medium flow, transceiver settings, and other functionality that would be apparent to a person of skill in the art.

FIG. 4 depicts an example of a flow sensor 400 that may be utilized in a medium measurement system. Such sensors are depicted, for example, in FIG. 2, as elements 208 and 226. The flow sensor 400 includes a housing 402 that defines a groove 404 that is configured to receive a conduit (e.g., downstream from an injector or upstream from a collection reservoir, as described elsewhere herein). Access to the groove 404 may be attained by lifting a cover 406. The sensor 400 may be disposable and/or parts of it may be disposable. The sensor 400 may also be re-useable and/or parts of it may be re-useable. As discussed, it may be important to provide the sensor 400 in a form that maintains the conduit/flow path integrity so as not to deflect or deform the environment in which it is housed (e.g., cover, groove, etc.). Insufficient rigidity may result in flow measurement errors. Although FIG. 3 depicts wired connections to the data acquisition box, the sensor 400 may be connected wirelessly and, as such, may include a transceiver 408 disposed in the housing 402. Furthermore, the firmware/software of the sensor 400 could also be part of the data acquisition unit and/or display, described elsewhere herein. In examples, the flow sensor 400 may be an ultrasonic sensor. In other examples, sensors may be utilized that do or do not have direct contact with the medium. Such sensors may also be utilized for the same purpose of implicitly or explicitly determining a volume injected into a patient.

Although a number of configurations of measurement and monitoring systems, as well as certain components used therein, are depicted in FIGS. 2-4, a number of other configurations, features, components, and functionalities are contemplated. With the above examples in mind, further examples are described in greater detail below. For example, a measuring and monitoring system may include an injector sensor module and a diverter sensor module that may include one or more ultrasonic flow sensors positioned about an injection fluid line out of the power injector and the medium diversion fluid line, respectively. The diversion fluid conduit sensor may be placed proximal or distal of the diversion valves. In examples, the diversion fluid sensor may be disposed within the diversion unit depicted, for example, in FIG. 3. In other examples, the diversion fluid sensor may be disposed external to the diversion unit, for example, proximate an inlet to the waste reservoir.

In another example, the power injector may incorporate a sensing element so as to determine the amount of medium ejected from the power injector. In such a case, the data information from this sensing element may be operatively connected to a processor to determine (in combination with the sensing data derived from medium diversion sensing module) the amount of medium injected into a patient. As described, the medium diversion line flow sensor may be positioned before, in proximity to, or after the diversion valves. Depending on the system construction, a location after the diversion valves could be advantageous by allowing the diversion sensor to be disposed in a non-sterile environment requiring less attention to the sterile field of the procedure. This would also potentially allow the diversion sensor to be reused for different patients and procedures. In another example, the diversion sensor may be incorporated into the collection container or waste reservoir, as described below.

The flow sensors described here may be custom designed to optimize flow measurement through the specific tube material and/or the diameter of the corresponding fluid conduit line. The sensor housings may be made from a polymer and may also include stainless steel or other rigid components so as to prevent the deflection/deformation of the measurement environment, which could introduce error into the flow measurements. Further, polymer, metal, or other non-porous housings may enable sanitization between patients. Each flow sensor may be calibrated, and an offset may be programmed into the sensor to increase the accuracy of the measurements. In other examples, sensor modules may be of a disposable, re-useable construction, or a combination of both.

Flow data obtained by the ultrasonic fluid flow sensors may be sent (via a wireless or wired connection) to a data acquisition unit, which may also include one or more processing devices to analyze the received signals, calculate diverted and delivered flows, identify error or other conditions, record diversion valve selections, etc. In an example, the data acquisition unit may be a FlowDAQ flow meter system, available from Strain Measurement Devices, Inc., of Wallingford, Connecticut. This or other flow meter systems from other manufacturers may include one or more receivers, circuit boards, processors, and data storage units to receive, process, and otherwise store and report results associated with the signals received from the various sensors included in the flow measurement system. In examples, any or all of the raw or processed data, error or other conditions, valve selections, etc., may be sent to a processing and/or display unit (e.g., an iPad, tablet, or other computing and/or display device). The information may be sent by electrical connection, or could be sent to the processor wirelessly, i.e., via Bluetooth, RFID, or other wireless connection. The FlowDAQ flow meter system, or other acquisition apparatus, may include firmware to convert the signal(s) from the flow sensors into actual flow and/or volume measurements, prior to sending the data to the processor and/or display.

As described previously, the flow sensor that is configured to measure the output of the power injector may be integral with the injector. The collected data or signals may be sent to a processor. The received or processed data and/or other information may be displayed on a display integral with the injector, or may be displayed at a single display that displays the various information relevant to the entire measurement system. In other examples, certain information relevant to the injector may be displayed at the injector, while certain information relevant to the entire flow measurement system may be displayed at the system display. In yet another example, the data acquisition unit may also be integral with the power injector system and injector sensor, simplifying the use of the system by reducing the number of remote fluid flow sensors positioned on the diversion conduit.

As an additional or alternative configuration, a character recognition device (CRD) may be utilized with the measuring system. In an example, it may be placed in proximity of the standalone display or the data information display of the power injector. The CRD may be used to obtain information as it relates to the amount of fluid being injected by the power injector, and other various conditions of the injector system. Information obtained from a scan may be sent to a data acquisition unit, display, or other relevant component for further analysis in determination of the fluid injected into the patient, as well as display of such interaction to a user.

The system may further include a stopcock, or other multi-way selection device for selecting one of the fluid diversion pathways. As noted above, while two fluid diversion pathways are generally depicted, multiple parallel pathways may be utilized. The stopcock, as well as other valves within the system, may have a sensor (e.g., Hall Effect, magnetic, electrical, pressure, fluid, ultrasonic, light, etc.) to monitor stopcock position of the diverter fluid flow path to the diversion valves. Each of the diversion valves may accommodate a range of pressures/flows to be modulated in the delivery of medium being injected into a patient. As an example, differing diversion valves may provide for differing delivery catheter configurations. For instance, one might have a flow range profile of a first divert valve to accommodate a 4 F/5 F delivery catheter (e.g., fluid conduit) configuration, while a second divert valve may better accommodate the use of a 6 F/7 F delivery catheter (e.g., fluid conduit) configuration. A third position of the stopcock could be an OFF position, closing the flow to either of the valves. In an exemplary configuration, a magnet may be positioned in, or in proximity of, the stopcock, providing a magnetic field in actuation of a Hall Effect sensor, thus identifying which of the diversion paths (through the at least two divert valves) is being used. Although this example includes a Hall sensor to identify the diversion pathway, many other technologies for sensing could be deployed including electrical, magnetic, acoustic, pressure, flow, etc. to identify the diversion pathway employed.

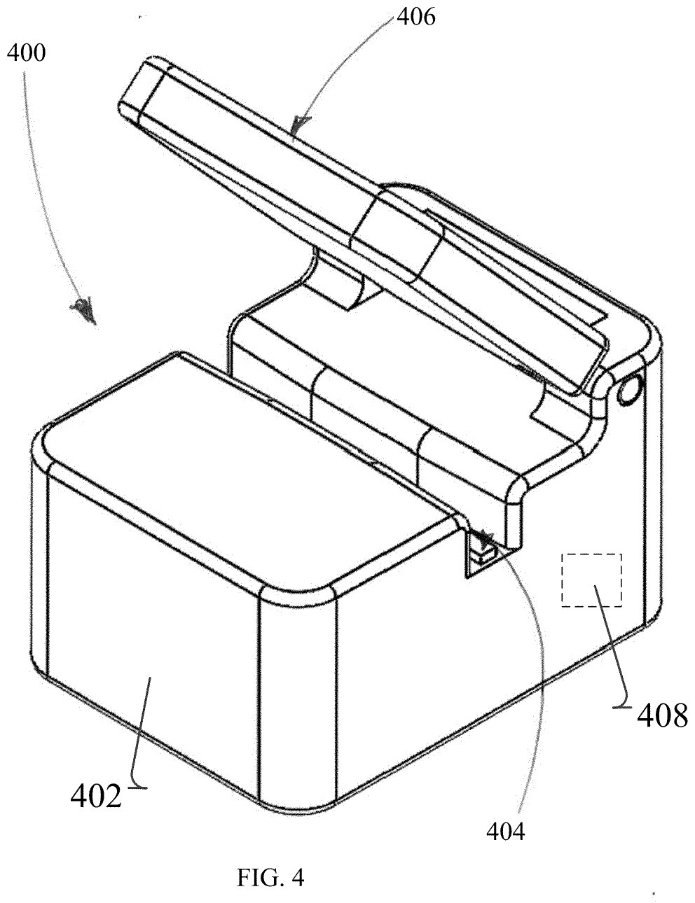

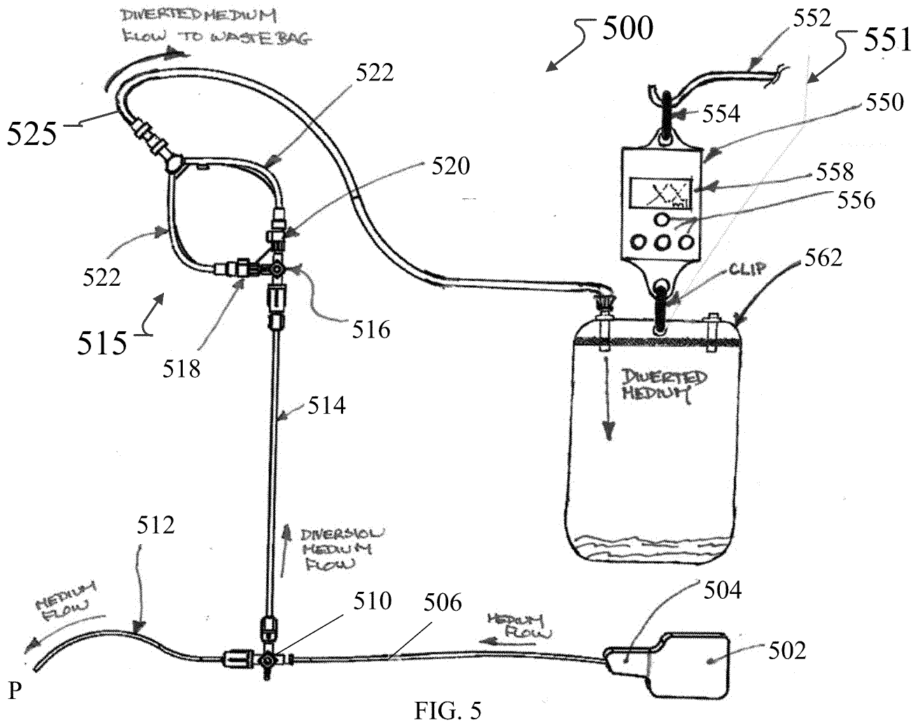

As stated previously, an example of measuring the divert line medium flow/volume via an ultrasonic sensor to the waste bag (collection container) may be accomplished through alternative sensing modalities, such a depicted in FIG. 5. This example includes replacing the ultrasonic sensor on the diversion line with a weight measurement on the waste bag/collection container, as described in more detail below. Thus, FIG. 5 illustrates an alternative sensing arrangement that may be used to measure the amount of medium that passes out of a fluid flow control apparatus 515 and into a waste bag or reservoir 562. As used herein, the term “waste” for the waste bag 562 is not to be limited to fluid medium that may be wasted, but rather to fluid medium that may be removed/diverted and then collected from being directly injected to a patient P during an injection. For example, it is contemplated that the medium captured may be re-used if it remains sterile. Certain features and components of the fluid measurement system 500 are consistent with those described in the injection/diversion systems depicted in FIGS. 1A-2, and as such, are not necessarily described further, or in greater detail.

FIG. 5 depicts an example of a fluid measurement system 500 that may be used with a power injector 502 so as to reduce the amount of medium injected into a patient P, while monitoring/measuring the total amount of medium. As shown, medium (e.g., contrast) may be supplied by the power injector 502 from a medium injection chamber 504 and into a discharge conduit 506. A portion of the medium injected by the injector 502 may be directed to the patient P via a catheter 512, while at least another portion of the medium may be simultaneously diverted through a diversion pathway 514. The flow of medium is indicated in FIG. 5 with arrows showing medium flow out of the injector chamber 504, flow to the catheter or patient conduit 512, flow to the diversion conduit 514, and the diverted medium conduit 525 into the waste bag or reservoir 562. FIG. 5 includes two diversion pathways 522 that modify the injection from the injector 502 to the patient P, although more than two could be used depending on the user needs. A divert valve selection toggle 516 may be used to select between the differing pathways 522, and the divert valves 518, 520 associated therewith. And, as described previously, the divert valves as flow restrictors 518, 520 may be constructed so as to provide differing pressure/flow modulating profiles that may address different use criteria (e.g., delivery catheter dimensions, arterial blood flow, etc.).

FIG. 5 depicts a system 500 that does not utilize a sensor on the conduit from the injector (as depicted elsewhere herein), but such a measurement device could still be employed in an alternative example. In this exemplary configuration, the injector 502 includes a measurement apparatus as is known in the art, so as to determine the amount of medium injected from the injector 502. FIG. 5 also depicts a collection and measuring apparatus 551 that includes a scale 550, or similar weight/mass sensing device disposed in proximity of a collection container 562, and at a terminal end of a waste conduit 525. The sensing device 550 may be hung from a bag holder 552 (such as an IV bag pole or like) and the waste bag/collection container 562 may be attached to the sensing device 550. These attachments may be constructed with easily detachable clips 554, rings, toggles, lines or the like, as shown in FIG. 5. Conversely, the bag 562 and the measuring/sensing device 550 may be integrally constructed as a single device. Measuring by the sensing device 550 may be performed by spring, force/strain, and hydraulic gauges, etc.

With the density of the fluid medium (quantity of mass per unit volume), the volume of medium in the bag 562 may be obtained. Conversely, correlation of weight/mass to the volume in the bag 562 may be empirically derived. As can be seen in FIG. 5, the sensing device 550 may include one or more buttons, toggles, switches, contacts (e.g., if a touch screen is utilized), or other controllers 556. A screen 558 or read-out is also depicted. The controllers 556 may be used for powering the device 550 on and off, taring, as well as selection purposes (e.g., the device 550 may include one or more given different medium densities (e.g., different agents, blending an agent with another fluid such as saline, etc.)). The scale 550 and collection bag 562 may be set-up and the scale 550 may be tared so as to remove any measurement of weight/mass of the bag 562 and other accessories (e.g., only the collected fluid medium may be measured). Moreover, measurements may be directly displayed as volume (e.g., ml, L) as a direct relationship to the sensor measurement. Further, measurements may be made in weight/mass and sent to a processor for calculations. In determining the amount of medium injected to the patient P, the amount/volume of medium diverted may be subtracted from the total amount/volume of medium injected by the injector 502. To this end, a surgeon or system user may simply read the two values from the readout 558 and determine the amount injected to the patient. Conversely, as described previously, there may be a variety of ways to send the information to be processed to a remote device (such as via wired or wireless connections). The processor may be disposed in a separate component (e.g., an iPad) or could be combined with the injector 502, and/or a measuring sensor device.

FIG. 5A depicts an alternative example of a sensing device 551a and waste receptacle 562a that may be utilized in a fluid measurement system. FIG. 5A makes clear that any container may be utilized to capture, trap or otherwise retain the diverted medium. An exemplary alternative construction of a collection and measuring apparatus 551a is depicted in FIG. 5A wherein the diverted medium may be directed via the waste conduit 525 to a container 562a, such as a beaker. This collected medium may be measured by a sensor device such as a scale 550a. Similar to the sensor device of FIG. 5, the sensing device 551a may have multiple buttons 556a to perform various functions as discussed previously. Results may be displayed on a screen or readout 558a.

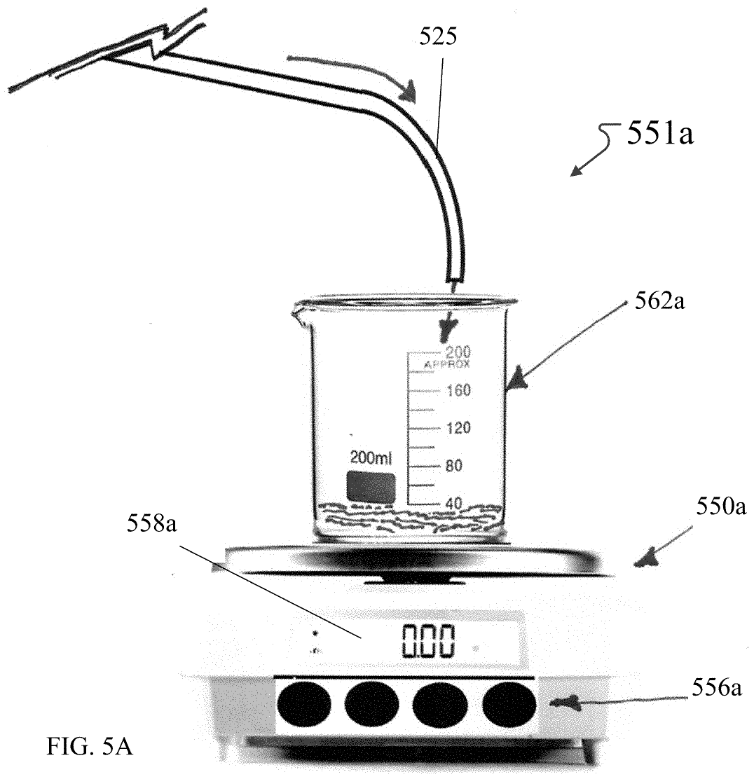

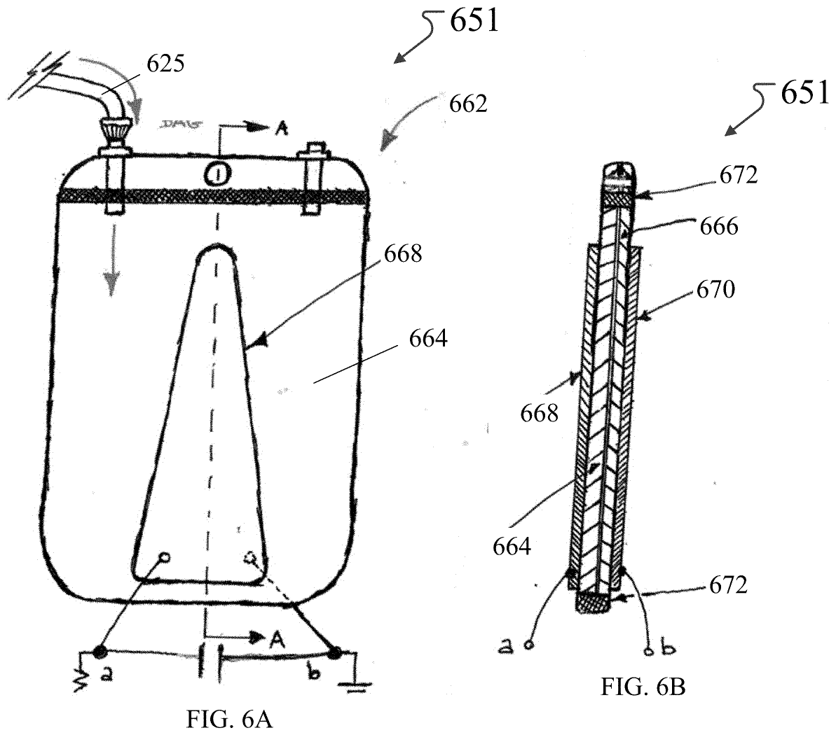

An alternative collection and measurement container 651 for measuring the volume of a fluid collected from a waste conduit 625 into a collection container 662, such as a waste bag, is depicted in FIGS. 6A and 6B. FIG. 6A is a front view of the collection container 662, and FIG. 5B is a cross-sectional view along A-A′. FIGS. 6A and 6B depict the collection container 662 having a form and/or function of a flexible and expandable waste bag, which may be incorporated into any of the other systems depicted elsewhere herein. Arrows indicate the medium flow from the waste conduit 625 into the collection container 662.

The collection container 662 employs structure thereon to enable sensing of the amount of medium in the collection container 662. The collection container 662 may include a flexible front wall 664 and a flexible rear wall 666, although examples with only a single flexible wall are contemplated. An electrically conductive element 668 may be secured to the front wall 664, and a similar electrically conductive element 670 may be secured to the rear wall 666. These elements 668, 670, may function as a capacitor between the front wall 664 and the rear wall 666 of the collection container 662. A capacitor is an electrical element that may be used to store energy by being “charged” and then discharged. Each of the elements 668, 670 may be a metallic foil, tape, film, print, or other electrically conductive material. As illustrated, the elements 668, 670 are applied to each wall 664, 666 of the collection container 662 (e.g., front and rear). In the example depicted, preferably each conductive element 668, 670 is relatively flexible so as not to significantly change the flexible properties of the front wall 664 and the rear wall 666. Moreover, an additional laminate, or other type material, may be placed over the elements 668, 670 so as to protect them from damage. Some materials that may be used for capacitor elements include: aluminum, silver, brass, copper, tantalum, carbon, titanium or other electrolytic capacitor material, one or more of which may be readily incorporated into the collection and measuring apparatus 651 depicted herein.

The material that forms the front wall 664 and the rear wall 666 (as well as any fluid contained therein) acts as a dielectric between the two electrolytic elements 668, 670, which act as the conductors of the capacitor. Terminals a and b in FIGS. 6A and 6B may be attached to the electrolytic elements 668, 670 in order to charge the capacitor (with a battery, not depicted). Terminals a and b may be available to take measurements of the strength of the capacitor. The front and rear walls 664, 666 are sealed at a plurality of seal locations 672. These seals may be formed by ultrasonic welds, adhesives, or liquid-tight mechanical fasteners. The location, configuration, thickness, and orientation of the seal locations may at least partially dictate expansion of the collection container 662.

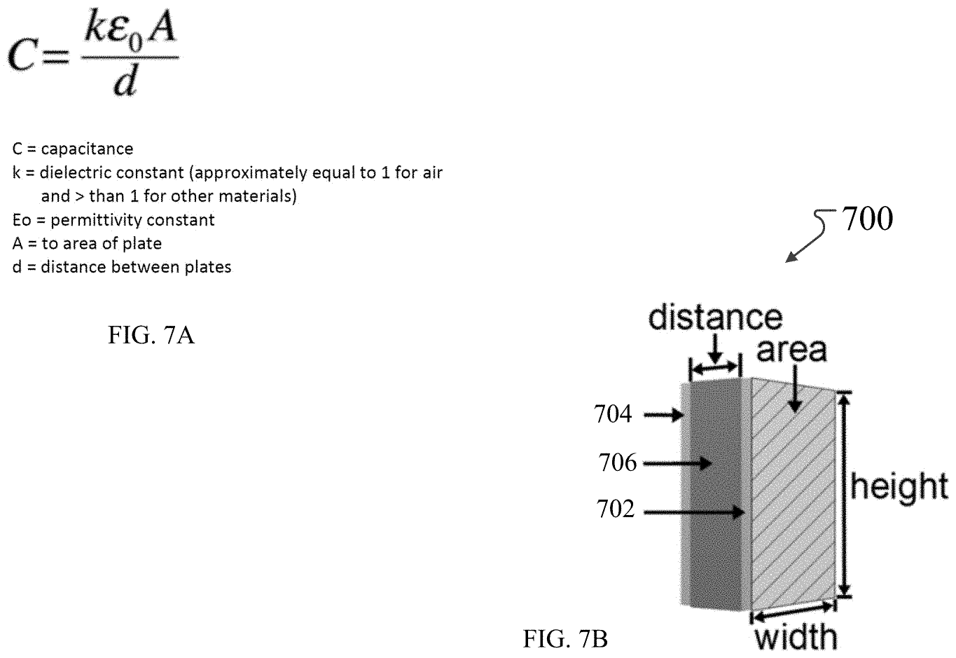

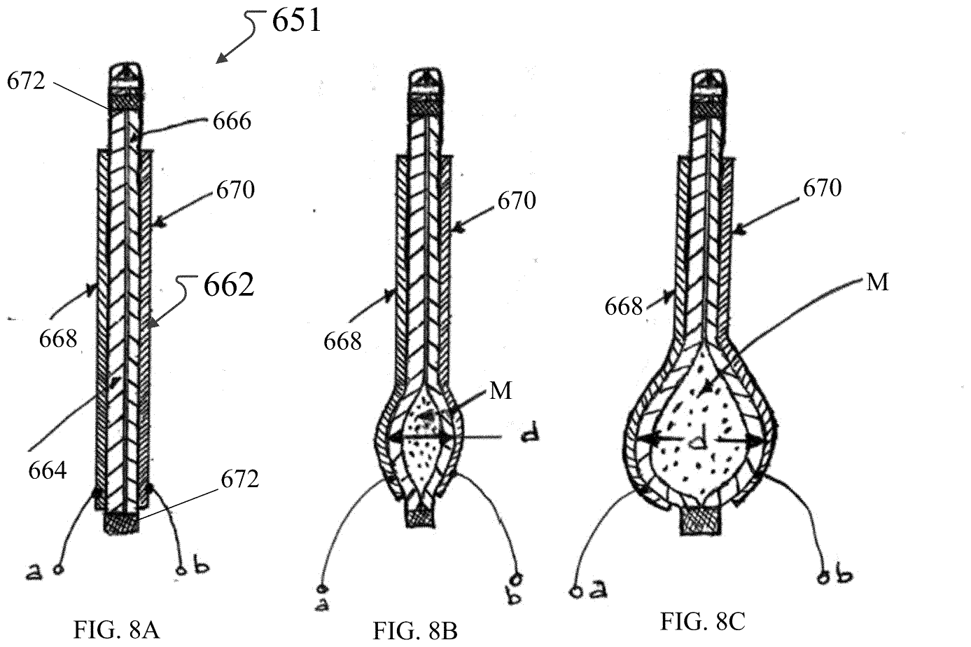

FIGS. 7A and 7B depict details regarding operation of a known capacitor, for illustrative purposes. The measure of how much electrical energy may be stored in a capacitor is measured as capacitance (Farads or Coulombs per Volt). The greater the Farads, the greater the capacity of the capacitor. The capacitance of a flat plate capacitor may be characterized by the equation in FIG. 7A. When in a substantially empty condition, collection and measurement apparatus 651 depicted in FIGS. 6A and 6B approximates the configuration of a flat plate capacitor 700, such as depicted in FIG. 6B. The front plate 702 corresponds with one electrolytic element 668, while the rear plate 704 corresponds with the other electrolytic element 670. These elements 668, 670 may be charged with a DC battery, or the charge to the capacitor may be varied, such as a square wave or sinusoidal wave. The front and rear walls 664, 666, as well as the fluid disposed therein, correspond to the dielectric 706 of the flat plate capacitor 700. As the collection and measuring apparatus 651 fills with medium, the front and rear walls 664, 666 may separate, increasing the value (e.g., distance) between the front and rear walls 664, 666, and thus the distance between the electrolytic elements 668, 670 (and, altering the capacitance thereof). This may enable a determination of the amount of fluid diverted to the collection container 662. This is further depicted in detail in FIGS. 8A-8C.

FIGS. 8A-8C depict a fluid collection and measurement container 651 of FIGS. 6A and 6B at various filled conditions, with distance d increasing from the substantially unfilled condition of FIG. 8A, to the partially-filled condition of FIG. 8B, to the more partially-filled condition of FIG. 8C. As may also be seen in FIGS. 8A-8C, the height of the fluid in the collection container 662 may not increase as rapidly as the container 662 bulging to accommodate the filling with medium M. Capacitance measurements may be made at various times of filling the collection container 662. Theoretically derived, or empirically obtained, data correlating volume in the collection container 662 with a change in capacitance may be determined. This data may be used to convert the change in capacitance measured to the volume of fluid within the collection container 662. Exact capacitance measurements may not need to be required to determine volume collected since the relative capacitance (Cᵣₑₗ) between two, or more, different time frames can simply be compared as to time frame one (t¹) and time frame two (or more), t². That is to say, the capacitance (A¹/d¹) at t¹ can be compared to capacitance at t², (A²/d²). The difference between these two values may be used to determine the empirically derived value of medium volume in the collection container 662. As the distance d depicted in FIGS. 8A-8C increases with the collection container 662 filling, capacitance measurements made between terminals a and b will decrease. Therefore (Cᵣₑₗ¹) when the collection container 662 is empty or substantially empty (e.g., FIG. 8A) is greater than the (Cᵣₑₗ²) when the collection container 662 is being filled (e.g., FIG. 8B, or FIG. 8C).

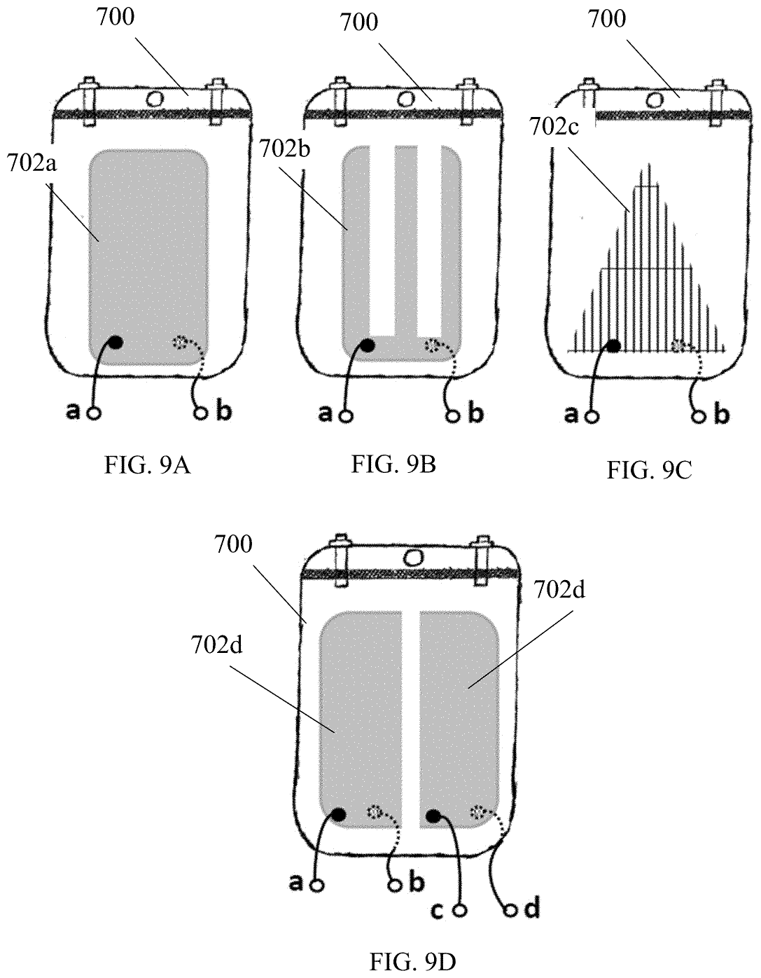

FIGS. 9A-9D depict collection containers 700 having alternative examples of sensing devices 702a, b, c, d. The sensing devices described generally herein may be configured to increase the reliability of measuring medium directed into the collection containers 700. As shown, the various shapes may allow for greater sensitivity to capacitance measurement at the bottom of the collection container 700, wherein the filling of medium may be greatest (and which may form a bulge in the collection container 700). Further, although two terminals a, b are depicted (e.g., one on a first conductive element, one on a second electrolytic element) multiple terminals may be used for taking capacitance measurements at various parts of the collection container 700. Sensing devices in the form of electrolytic elements having varied forms are depicted in FIGS. 9A-9C. These configurations depict sensing devices (702a, 702b, 702c) having a single set of terminals, with terminal a affixed to the one side and terminal b affixed to the opposite side. Additional electrolytic form constructions are contemplated. The various, different constructions may improve measurement quality, reliability, sensitivity and/or reduce product costs, to name a few advantages of various configurations. FIG. 9D depicts a construction of two sensing devices 702d attached to the collection container 700. Front terminals a and c are shown, along with back terminals b and d. Multiple capacitor constructions with two or more terminal sets may be used to improve the performance of the sensing devices depicted herein (e.g., 702d of FIG. 9D). In general, FIG. 9A depicts a rectangular element 702a. FIG. 9B depicts an irregularly-shaped element 702b. FIG. 9C depicts a crisscrossing electrolytic element 702c. The elements 702d of FIG. 9D are generally rectangular, and have multiple terminal sets (e.g., a-b, c-d). The depicted configurations are not intended to be exclusive of other constructions, but rather they are intended to be exemplary. The various element configurations and the number and placement of terminals may be modified as required or desired to increase the reliability of the capacitance measurements. Multiple terminal sets may help in checking or qualifying measurements against one another, and/or may be used to assure that a container is filling evenly (e.g., level and/or correcting for a differing filling manner).

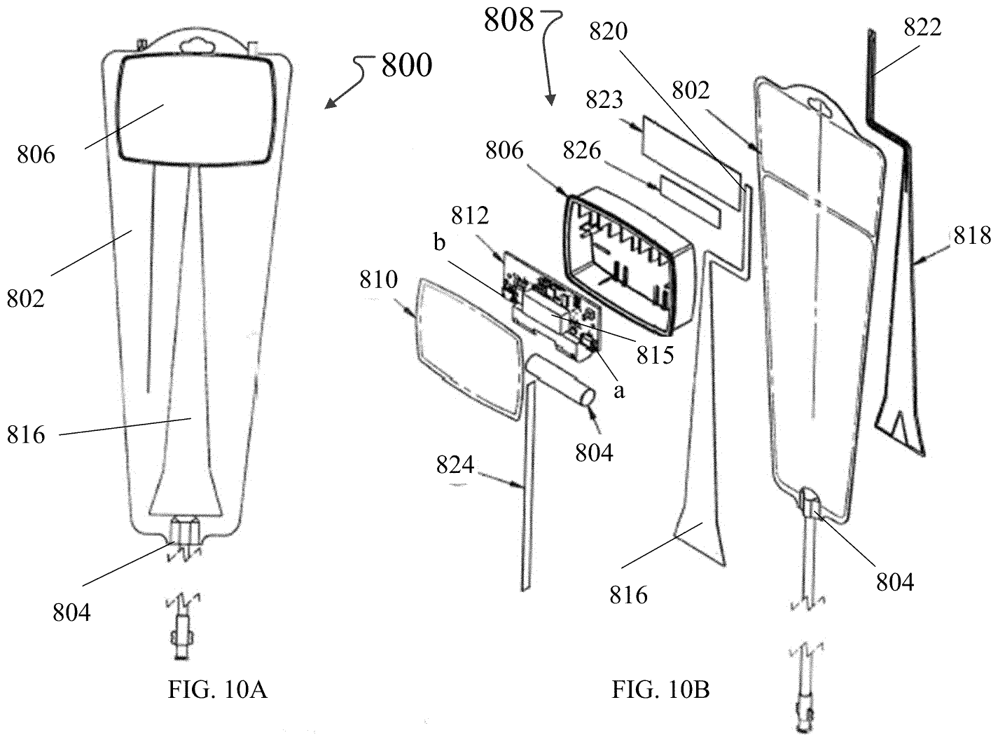

FIGS. 10A and 10B depict front and exploded perspective views of an alternative example of a sensing device 800 utilized on a collection container 802 that in this case is in the form of a flexible bag. Examples of the technology depicted above include collection containers where the diverted medium is delivered at a top opening of the collection containers. In the example of FIGS. 10A and 10B, diverted fluid is introduced to the collection container 802 proximate at an inlet 804 disposed proximate a bottom portion of the collection container 802, mostly below elements of the sensing device 800. Such a configuration may be utilized to improve the performance of the sensing device 800, as diverted fluid may more easily pool at the bottom of the collection container 802, at its point of introduction. Configurations utilizing an inlet on the side, top, or other entrance of a collection container are also contemplated.

Other configurations of collection containers are contemplated, such as different shapes (which may result in differing volumes at various locations within the collection container). These differing configurations may improve the total performance of the measuring device. Returning to FIG. 10A, an alternative collection container 802 is depicted that includes an inverted, generally tapered shape. This configuration results in a smaller fluid capacity proximate a bottom portion of the collection container 802, with a greater fluid capacity proximate an upper portion. As shown, the fluid may enter the collection container 802 at the inlet 804 disposed at the bottom thereof. As with other collection containers depicted elsewhere herein, this collection container 802 is formed of flexible materials, and may be hung from a support disposed above the collection container. When hanging, the bottom of the collection container 802 is closest to the floor. FIG. 10B depicts an exploded perspective view of the collection bag 802 and a housing 806 disposed thereon.

As illustrated in the FIGS. 10A and 10B, the collection container 802 may have secured thereto a measurement apparatus 808, components of which may be disposed in the housing 806. The housing 806 may be sealed by a cover 810. The measurement apparatus 808 may include a circuit board 812 disposed in the housing 806. Various components disposed on the circuit board 812 may include capacitance measurement circuitry and a processor, terminal a and terminal b, battery 814, and a signal generator 815 (for example, for communicating with an associated tablet, computer, display, or other devices as described herein). In examples, the housing 806 need not be affixed to the collection bag 802, but is only depicted affixed for illustration. Further, the housing 806 and its cover 810 may be constructed in other form factors, may be liquid tight, and may include one or more light emitting elements, displays, buttons, switches, etc. disposed thereon.

A front conductive element 816 and a rear conductive element 818 are also depicted, and they include a generally triangular form factor. In examples, the elements 816, 818 may be carbon based, or other materials as previously discussed, and may be printed onto the collection container 802. An end 820 of electrolytic element 816 may be passed through the back of housing 806 and applied to terminal a on the circuit board 812. Similarly, an end 822 of the electrolytic element 818 may be passed through collection container 802, then may be routed through a rear of the housing 806 and affixed to terminal b of the circuit board 812. The battery 814 may be used to power a processor and circuit measurements of the circuit board 812, the signal generator 815 to display measurement information, and any other components that may require power, such as a wireless/Bluetooth to transmit the data/information.

Further, the battery 804, signal generator 815, measure for capacitance, wired/wireless connection, data collection and processor on the circuit board 812 may be integrally combined with the collection container 802 and may include a read-out 815, or other display. Double-sided adhesive tape 823, 826 or other fixation/bonding material, illustratively shown, may allow for affixation of measurement unit 808 to bag 802.

Conversely, these components may be a part of other components in the system (e.g., the processor residing in an associated iPad, for example). A pull tab 824 may be disposed between one of pole connections of the battery 814, which may allow for the battery 814 to be in place (e.g., during storage, shipment, etc.) but not powered-up until it is pulled/removed from the battery pole connection.

The process of measuring capacitance maybe accomplished by periodically applying a voltage across the capacitance sensor (elements 816, 818) and measuring the time needed for the charge on the sensor to reach the applied voltage level. A resistor maybe included in-series with the capacitance sensor on the circuit board 812, forming a resistor-capacitor (RC) network, thus slowing the charge/discharge time enough to allow accurate measurements to be made by the sensor. In practice, a voltage level on the sensor may be measured after fixed time duration from start of charging. In this case the measured voltage level may be proportional to current sensor capacitance. Further, techniques to measure a capacitance by charging the RC sensor to a higher voltage may have a similar affect as discharging the RC sensor to a lower voltage.

On circuit board 812, RC sensor charging may be accomplished by using a switching transistor, or CPU output pin, that may change the voltage on the RC network sensor, as needed. Further, an analog-to-digital converter could be used to measure voltage values on the sensor itself (thus, bypassing the resistor). By analyzing data related to charge times and voltages, the capacitance of the sensor can be calculated by a microcontroller. Current sensor capacitance determined with charge/discharge times may be used to determine how much fluid resides within the collection container 802 (mathematically and/or empirically). Other circuit schemes (i.e., to improve performance, reduce costs, increase quality, for example) may be used to measure capacitance and this is only illustrative of a way to measure fluid in a collection container 802.

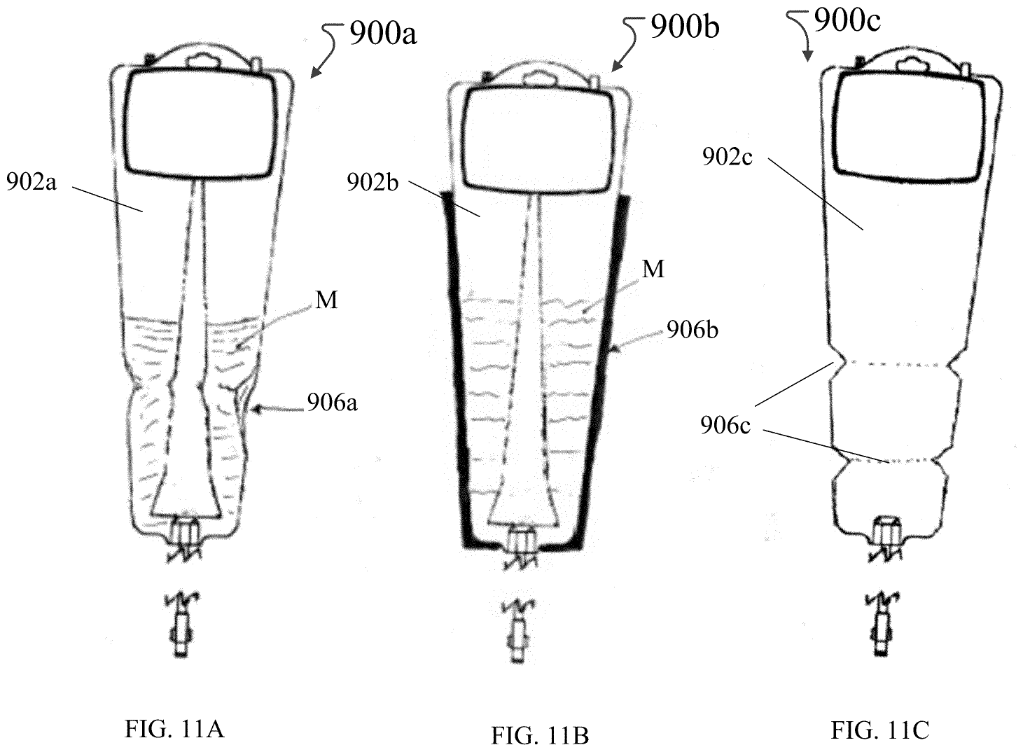

As discussed previously, there may be many different shapes and sizes to the collection containers, as well as they may be of a variety of structural formations. The various shapes, sizes, and structural elements may be used to optimize the performance, reliability, cost, quality, etc. of the collection container. FIGS. 11A-11C depict such alternative examples of sensing devices 900a-c and collection containers 902a-c. FIG. 11A depicts a collection container similar to that depicted in FIGS. 10A-10C. As the collection container 902a is filled with liquid (e.g., medium M), a bulge 906a may form. If this bulge 906a does not form in the same place each time the collection container 902a is filled, capacitance measurement errors may be encountered. As such, in one example, expansion control features may include a collection container 902a expanding equally/reliably each time it is filled. FIG. 11B depicts one such modified collection to container 902a. Container 902b may include an “exoskeleton”, or other support structure 906b, which may maintain the shape of the collection container 902b, and may help ensure equal, and reliable, expansion of the container 902b. The exoskeleton 906b may be a relatively rigid support positioned surrounding the bag that forms the collection container 902b, or may include other structural elements (e.g., ribs, battens, clips, clasps, rigid positions of the container, etc.). Thus, the exoskeleton 906b (or, rigid structure surrounding the bag) may help reduce measurement error. In another example, depicted in FIG. 11C, the collection container 902c may be shaped so as to improve filling consistency, and reduce random bulges 906a while filling. Another embodiment to help reduce errors (in filling collection bag measurements as a result of bag bulges), can be seen in FIG. 11C. A shown in FIG. 11C, he collection container 902c may include pre-formed bulges, creases, or other features 906c that cause the collection container 902c to deform consistently each time it is filled. Note that, for simplicity, the capacitance plates and fluid are not shown in FIG. 11C.

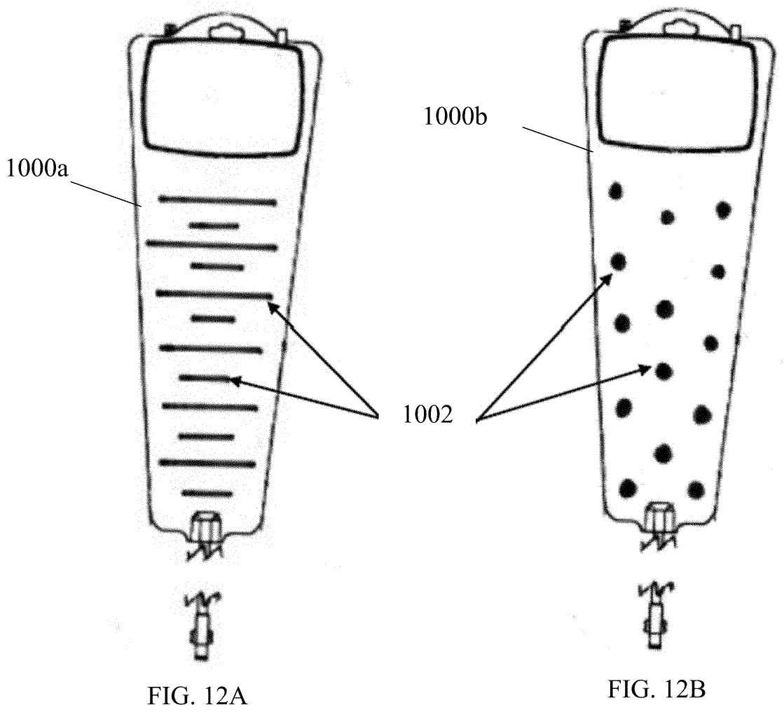

FIGS. 12A-12B depict alternative examples of collection containers 1000a (front side), 1000b (back side). The collection containers 1000a, 1000b may include expansion control features 1002 in the form of interior bridges between the front and rear walls of the containers 1000a, 1000b (as opposed to the portions at the edges, or areas that might be reinforced between the front and rear walls that defining containers 1000a, 1000b). The interior bridges 1002 may be formed by sealing, bonding, melting, molding, fixating, adhering, and/or otherwise matting portions of the front and back walls of the flexible material that forms the collection containers 1000a, 1000b together. FIGS. 12A and 12B indicate examples of where the front and rear of the collection containers 1000a, 1000b may be affixed together, thus these elements may provide more structure to the collection containers 1000a, 1000b and increase reliability during filling of the collection containers 1000a, 1000b.

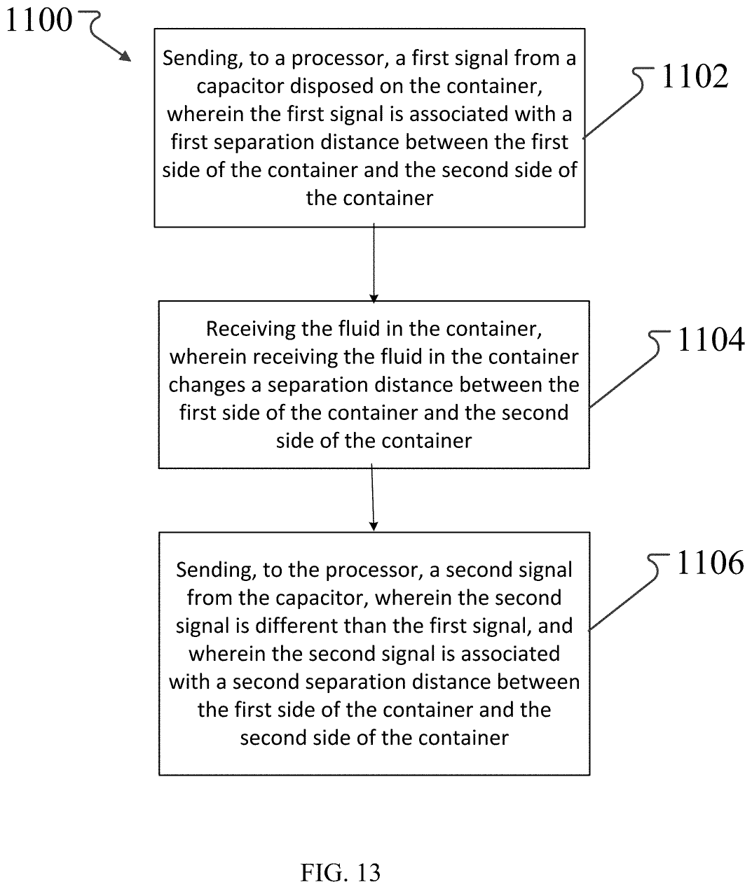

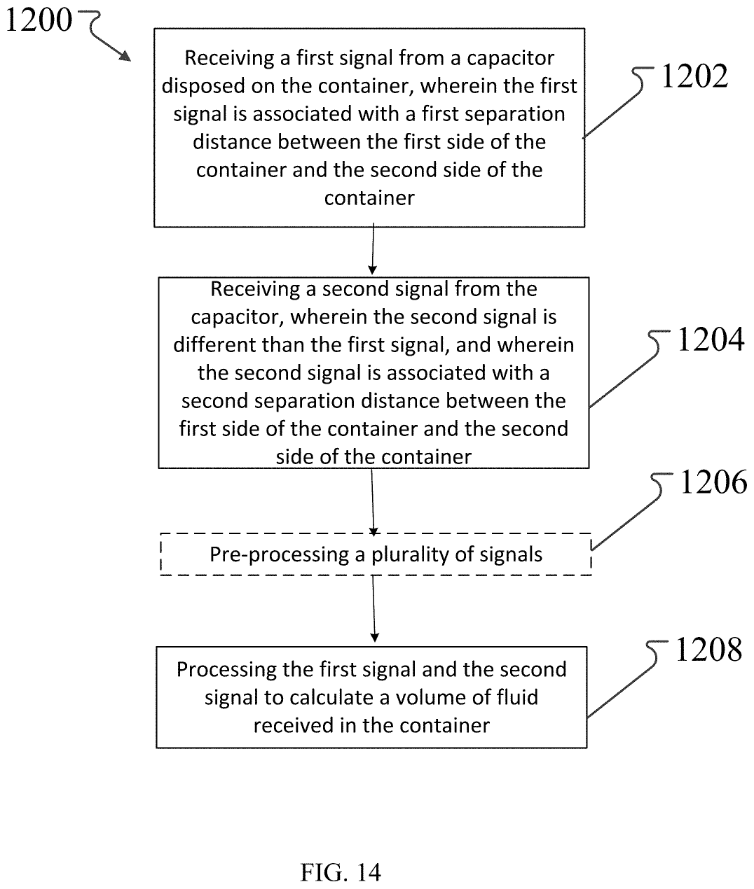

FIG. 13 depicts a method 1100 of determining a volume of a fluid in a container. The container may be any of the collection and measurement containers depicted and described herein, for example. In examples, the container includes a first side and a second side adjacent the first side. The method 1100 begins with sending first signal from a capacitor disposed on the container, operation 1102. The first signal may be sent to a processor, for example, of the data acquisition unit or the tablet associated therewith. The first signal is associated with a first separation distance between the first side of the container and the second side of the container. In that case, the first signal is indicative of a first capacitance measurement, e.g., of the material of the collection container, as well as any fluid that may be disposed therebetween. In operation 1104, fluid is received in the container. This receipt of fluid changes a separation distance between the first side of the container and the second side of the container. In operation 1106, a second signal is sent from the capacitor, again to a remote processor. The second signal is different than the first signal and is associated with a second separation distance between the first side of the container and the second side of the container. In examples, the first signal is a plurality of first signals and the second signal is a plurality of second signals. These pluralities of signals may correspond to signals sent from multiple terminals located on the capacitive elements, for example, as depicted elsewhere herein. At the processor, they may be processed consistent with techniques described with regard to FIG. 14, described below.

FIG. 14 depicts a method 1200 of calculating a volume of a fluid in a container. The container may be any of the collection and measurement containers depicted and described herein, for example. In examples, the container includes a first side and a second side adjacent the first side. The method 1200 begins with receiving a first signal from a capacitor disposed on the container, operation 1202. The first signal is associated with a first separation distance between the first side of the container and the second side of the container. In operation 1204, a second signal is received from the capacitor. The second signal is different than the first signal and is associated with a second separation distance between the first side of the container and the second side of the container. In operational operation 1206, pre-processing of a plurality of signals may be performed. In certain examples, where multiple terminals are utilized on a since capacitive element, the first signal includes a plurality of first signals and the second signal includes a plurality of second signals. Pre-processing of such signals may include calculating an average value of the plurality of second signals, calculating a standard deviation of the plurality of second signals, calculating a median value of the plurality of second signals, and/or associating a one second signal of the plurality of second signals with a one first signal of the plurality of first signals. By utilizing multiple signals, a more detailed measure of the fluid contained within the collection container may be obtained, allowing or a more accurate calculation of fluid injected into the patient. Pre-processing may result in a single resultant signal, or a plurality of signals, depending on the type of pre-processing performed. In operation 1208, the first signal and the second signal are processed to calculate a volume of fluid received in the container, which can then be used to obtain the amount of fluid introduced to the patient. In examples where a resultant signal is maintained, processing the first signal and the second signal contemplates processing the resultant signal.

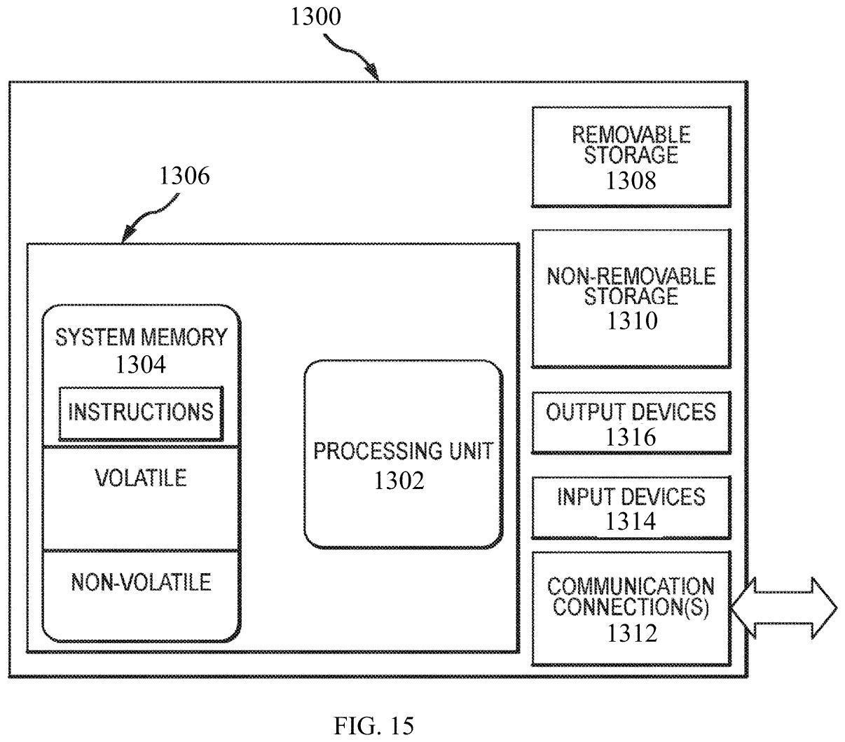

FIG. 15 illustrates one example of a suitable operating environment 1300 in which one or more of the present embodiments may be implemented. This is only one example of a suitable operating environment and is not intended to suggest any limitation as to the scope of use or functionality. Other well-known computing systems, environments, and/or configurations that may be suitable for use include, but are not limited to, personal computers, server computers, hand-held or laptop devices, multiprocessor systems, microprocessor-based systems, programmable consumer electronics such as smart phones, network PCs, minicomputers, mainframe computers, smartphones, tablets, distributed computing environments that include any of the above systems or devices, and the like. In an example, the operating environment 1300 may be the data acquisition unit depicted herein, the display, the power injector, or combinations of several components.

In its most basic configuration, operating environment 1300 may typically include at least one processing unit 1302 and memory 1304. Depending on the exact configuration and type of computing device, memory 1304 (storing, among other things, instructions to perform the calculating and measuring methods described herein) may be volatile (such as RAM), non-volatile (such as ROM, flash memory, etc.), or some combination of the two. This most basic configuration is illustrated in FIG. 13 by line 1306. Further, environment 1300 may also include storage devices (removable, 1308, and/or non-removable, 1310) including, but not limited to, magnetic or optical disks or tape. Similarly, environment 1300 may also have input device(s) 1314 such as touch screens, keyboard, mouse, pen, voice input, etc. and/or output device(s) 1316 such as a display, speakers, printer, etc. Also included in the environment may be one or more communication connections, 1315, such as LAN, WAN, point to point, Bluetooth, RF, etc.

Operating environment 1300 may typically include at least some form of computer readable media. Computer readable media can be any available media that can be accessed by processing unit 1302 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, solid state storage, or any other tangible medium which can be used to store the desired information. Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The operating environment 1300 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet. In some embodiments, the components described herein comprise such modules or instructions executable by computer system 1300 that may be stored on computer storage medium and other tangible mediums and transmitted in communication media. Computer storage media includes volatile and non-volatile, removable and non-removable media implemented in any method or technology for storage of information such as computer readable instructions, data structures, program modules, or other data. Combinations of any of the above should also be included within the scope of readable media. In some embodiments, computer system 1300 is part of a network that stores data in remote storage media for use by the computer system 1300.

The technologies described herein provide decided advantages over prior art systems, devices, and methods due to their simplicity of construction, control, and operation. The more expensive components of the measurement and monitoring systems described herein (e.g., the ultrasonic flow detectors, data acquisition device, and powered injector) may be reusable. The various conduits (e.g., from the injector, diversion medium flow, waste, and to patient), valves, and stopcocks may be disposable. As such, the components that are or may be potentially exposed to patient bodily fluids may simply be disposed of after use. This avoids the necessity for cleaning or sanitizing between patients or, worse, the risk of cross-contamination.

Although various types of sensors may be utilized, it has been determined that the use of ultrasonic sensors might be particularly desirable, since such sensors display particularly fine resolution at the low flow rates and volumes typical in the contemplated medium injection systems. For example, flow rates at the injector outlet or prior to the waste container may be from about 0.5 ml/sec to about 20 ml/sec at the extremes. At such flow rates, ultrasonic sensors may provide the most accurate measurements available, thus helping to ensure accurate measurement of fluid injected to the patient. Further, the ultrasonic sensors need not penetrate the various conduits (e.g., unlike certain other flow detectors), thus eliminating additional potential contamination points.

The power injector may display particular advantages over manually-operated devices, such as syringes. The controls of the injector may be set in advance to inject at a desired flow rate, pressure, or other condition as required or desired for a particular application. Thus, with the injection parameters preset into the injector controller, the surgeon may be free to monitor or control other aspects of the procedure to ensure a desirable result. In examples, the power injector may be incorporated into a stand-alone device with one or more of the data acquisition unit, injector sensor, and data processor and display. Thus, a single remote diversion sensor may communicate with the combined system and simplifying system set up and operation.

The data acquisition system (whether or not integrated with the power injector) may also be programmed with, for example, the dimensions (e.g., length and lumen size) of the various conduits, the volume of the various conduits, positions of the sensors along the various conduits, or other system specifications so as to improve accuracy. Error conditions (such as matching fluid flows at both the injector sensor and the diversion sensor may be indicative of an obstructed patient conduit, thus triggering a warning or other error condition. Other unexpected discrepancies or significant deviations between signals sent from the various sensors may be indicative of other problematic system conditions that may require termination of the procedures being performed.

While there have been described herein what are to be considered exemplary and preferred examples of the present technology, other modifications of the technology will become apparent to those skilled in the art from the teachings herein. The particular methods of manufacture and geometries disclosed herein are exemplary in nature and are not to be considered limiting. It is therefore desired to be secured all such modifications as fall within the spirit and scope of the technology. Accordingly, what is desired to be secured by Letters Patent is the technology as defined and differentiated herein, and all equivalents.

The systems described herein are directed generally to measurements of a fluid medium, e.g., after being utilized with an injection/diversion system. The measurement of a fluid into a container, such as described herein, may be important in the management, diagnosis, and/or treatment of a varieties of diseases, diagnosis, and conditions. To this end, it is contemplated that the measurement devices may be helpful in determining volumes collected as part of procedures on patients in urology, neurology, cardiology, gynecology, oncology, hematology, bone related (ortho), to name only a few medical areas.

1. A fluid collection and measurement container comprising:
a first wall;
a second wall disposed opposite the first wall, wherein the second wall is secured to the first wall and wherein first wall and the second wall define an expandable volume therebetween;
a first electrolytic element disposed on the first wall;
a second electrolytic element disposed on the second wall;
a first terminal connected to the first electrolytic element; and
a second terminal connected to the second electrolytic element.

2. The fluid collection and measurement container of claim 1, wherein at least one of the first wall and the second wall is flexible.

3. The fluid collection and measurement container of claim 1, wherein the first electrolytic element comprises a foil.

4. The fluid collection and measurement container of claim 1, further comprising an inlet.

5. The fluid collection and measurement container of claim 4, wherein the inlet is disposed below the first electrolytic element and the second electrolytic element.

6. The fluid collection and measurement container of claim 4, wherein the fluid collection and measurement container comprises a tapered form factor, and wherein the inlet is disposed proximate a shorter side of the tapered form factor.

7. The fluid collection and measurement container of claim 1, wherein the first wall is secured to the second wall via an edge seal comprising at least one of an ultrasonic weld, an adhesive, and a mechanical fastener.

8. The fluid collection and measurement container of claim 7, wherein the edge seal comprises the mechanical fastener, wherein the mechanical fastener is liquid-tight.

9. The fluid collection and measurement container of claim 1, further comprising an expansion control feature, wherein the expansion control feature comprises at least one of an exoskeleton, a bulge, a crease, and a bridge.

10. The fluid collection and measurement container of claim 1, wherein the first terminal comprises a plurality of first terminals and the second terminal comprises a plurality of second terminals.

11. A system for measuring an amount of a fluid medium, the system comprising:
a power injector for providing automated ejection of the fluid medium;
a delivery catheter for providing delivery of at least a first portion of the fluid medium into the patient, during use;
at least two flow controllers selectively fluidically coupled to the power injector;
a fluid flow control apparatus fluidly coupled between the power injector, the delivery catheter, and the at least two flow controllers, wherein the fluid flow control apparatus, during use, provides fluid diversion of at least a second portion of the fluid medium, the second portion of the fluid medium being diverted away from the delivery catheter, and wherein an amount of diversion of the second portion of fluid is dependent on a selection of one of the at least two flow controllers, wherein the at least two flow controllers are characterized by applying differing resistances to the second portion of the fluid medium;
a collection container for receiving the second portion of the fluid medium;
first sensor capable of measuring an elected amount of the fluid medium ejected by the power injector; and
a second sensor disposed on the collection container, wherein the second sensor comprises a first part disposed on a first wall of the collection container, a second part disposed on a second wall of the collection container, and wherein the second sensor detects a change in capacitance between the first part and the second part.

12. The system of claim 11, wherein each of the at least two diversion valves are disposed on a discrete diversion pathway, and wherein the fluid flow control apparatus comprises a stopcock fluidically coupled to the power injector, wherein the stopcock is positionable in a first position and a second position, wherein when in the first position, a first diversion pathway associated with a first one of the at least two flow controllers is fluidically coupled to the power injector, and wherein when in the second position, a second diversion pathway associated with a second one of the at least two flow controllers is fluidically coupled to the power injector.

13. The system of claim 12, wherein the fluid control apparatus comprises a housing and wherein the at least two flow controllers are disposed in the housing.

14. The system of claim 13, wherein the stopcock is disposed within the housing and wherein the stopcock is manually positionable from an exterior of the housing.

15. The system of claim 13, wherein the fluid control apparatus further comprises a plurality of first light emitting elements disposed proximate the first diversion pathway and a plurality of second light emitting elements disposed proximate the second diversion pathway.

16. The system of claim 15, wherein the first plurality of light emitting elements and the second plurality of light emitting elements are selectively activatable based at least in part on a position of the stopcock.

17. The system of claim 11, further comprising a data acquisition unit communicatively coupled to the first sensor and the second sensor, wherein the data acquisition unit is configured to calculate the first portion of fluid based at least in part on an ejection signal received from the first sensor and a diversion signal received from the second sensor.

18. The system of claim 17, further comprising a display communicatively coupled to the data acquisition unit.

19. The system of claim 18, wherein the display is integral with the power injector.

20. The system of claim 11, wherein the collection container comprises an expandable collection volume.

22. The system of claim 11, wherein at least one of the first wall and the second wall is flexible.

23. The system of claim 11, wherein the first part of the second sensor and the second part of the second sensor comprises a capacitive foil.

24. The system of claim 23, further comprising a first terminal connected to the first part of the second sensor and a second terminal connected to the second part of the second sensor.

25. A method of determining a volume of a fluid in a container having a first wall and a second wall adjacent the first wall, the method comprising:
sending, to a processor, a first signal from a capacitor disposed on the container, wherein the first signal is associated with a first separation distance between the first wall of the container and the second wall of the container;
receiving the fluid in the container, wherein receiving the fluid in the container changes a separation distance between the first wall of the container and the second wall of the container; and
sending, to the processor, a second signal from the capacitor, wherein the second signal is different than the first signal, and wherein the second signal is associated with a second separation distance between the first wall of the container and the second wall of the container.

26. The method of claim 25, wherein the first signal comprises a plurality of first signals, and wherein the second signal comprises a plurality of second signals.

27. A method of calculating a volume of a fluid received in a container having a first wall and a second wall adjacent the first wall, the method comprising:
receiving a first signal from a capacitor disposed on the container, wherein the first signal is associated with a first separation distance between the first wall of the container and the second wall of the container;
receiving a second signal from the capacitor, wherein the second signal is different than the first signal, and wherein the second signal is associated with a second separation distance between the first wall of the container and the second wall of the container; and
processing the first signal and the second signal to calculate the volume of the fluid received in the container.

28. The method of claim 27, wherein the first signal comprises a plurality of first signals and wherein the second signal comprises a plurality of second signals, and wherein processing the first signal and the second signal comprises pre-processing the plurality of first signals and the plurality of second signals.

29. The method of claim 28, wherein pre-processing the plurality of second signals comprises at least one of:
calculating an average value of the plurality of second signals;
calculating a standard deviation of the plurality of second signals;
calculating a median value of the plurality of second signals; and
associating a first one of the plurality of second signals with a first one of the plurality of first signals.

30. The method of claim 29, wherein pre-processing the plurality of second signals results in at least one resultant second signal.

31. The method of claim 30, wherein processing the first signal and the second signal comprises processing the first signal and the resultant second signal.
